(19) United States
(12) Patent Application Publication
(10) Pub. No.: US 2003/0003496 A1
(43) Pub. Date: Jan. 2, 2003

(54) Novel compositions and methods for array-based nucleic acid hybridization

(75) Inventors: Allan Bradley, Cambridge (GB); Wei-Wen Cai, Pearland, TX (US)

Correspondence Address: GREGORY P. EINHORN, Fish & Richardson P.C., Suite 500, 4350 La Jolla Village Drive, San Diego, CA, 92122, US

(73) Assignee: Baylor College Medicine, a Texas corporation

(21) Appl. No.: 10/207,440

(22) Filed: Jul. 26, 2002

Related U.S. Application Data
(62) Division of application No. 09/839,658, filed on Apr. 19, 2001.
(60) Provisional application No. 60/210,153, filed on Jun. 7, 2000.

(51) Int. Cl. C12Q 1/68; C12P 19/34

(52) U.S. Cl. 435/6; 435/91.2

(57) ABSTRACT

The invention provides compositions and methods for generating a molecular profile of genomic DNA by hybridization of labeled nucleic acid representing the genomic DNA to immobilized nucleic acid probes, e.g., arrays or biochips.

### STATEMENT AS TO FEDERALLY SPONSORED RESEARCH

This invention was made with Government support under a grant from National Institutes of Health, No. R21 CA83211. The Government may have certain rights in this invention.

### TECHNICAL FIELD

This invention relates to molecular biology, genetic diagnostics and nucleic acid array, or “biochip,” technology. In particular, the invention provides novel methods and compositions for array-based nucleic acid hybridizations.

### BACKGROUND

Genomic DNA microarray based comparative genomic hybridization (CGH) has the potential to solve many of the limitations of traditional CGH method, which relies on comparative hybridization on individual metaphase chromosomes. In metaphase CGH, multi-megabase fragments of different samples of genomic DNA (e.g., known normal versus test, e.g., a possible tumor) are labeled and hybridized to a fixed chromosome (see, e.g., Breen (1999) J. Med. Genetics 36:511-517; Rice (2000) Pediatric Hematol. Oncol. 17:141-147). Signal differences between known and test samples are detected and measured. In this way, missing, amplified, or unique sequences in the test sample, as compared to “normal,” can be detected by the fluorescence ratio of normal control to test genomic DNA. In metaphase CGH, the target sites (on the fixed chromosome) are saturated by an excess amount of soluble, labeled genomic DNA.

In contrast to metaphase CGH, where the immobilized genomic DNA is a metaphase spread, in array-based CGH method the immobilized nucleic acids are arranged as an array, on, e.g., a biochip or a microarray platform. Another difference is that in array-based CGH the immobilized genomic DNA is in molar excess as compared to the copy number of labeled (test and control) genomic nucleic acid. Under such conditions, suppression of repetitive genomic sequences and cross hybridization on the immobilized DNA is very helpful for reliable detection and quantitation of copy number differences between normal control and test samples. However, when traditional protocols are used such suppression is less than optimal. Furthermore, genomic DNA is a promiscuous mix containing more than 30% repetitive sequences and a further unknown proportion of closely related sequences. These sequences can cross-hybridize when traditional protocols are used to prepare test and sample DNA for hybridization to the array.

### SUMMARY

The invention provides a method for generating a molecular profile of genomic DNA by hybridization of a genomic DNA target to an immobilized nucleic acid probe, comprising the following steps: (a) providing a plurality of nucleic acid probes comprising a plurality of immobilized nucleic acid segments; (b) providing a sample of target nucleic acid comprising fragments of genomic nucleic acid labeled with a detectable moiety, wherein each labeled fragment consists of a length smaller than about 200 bases; and (c) contacting the genomic nucleic acid of step (b) with the immobilized probes of step (a) under conditions allowing hybridization of the target nucleic acid to the probe nucleic acid.

In alternative embodiments, each labeled fragment consists of a length no more than about 175 bases; 150 bases; about 125 bases; about 100 bases; about 75 bases; about 50 bases; about 40 bases; about 30 bases; and about 25 bases. In another embodiment, each labeled fragment consists of a length between about 25 to about 30 bases and about 100 bases. These samples of target genomic nucleic acid can prepared using a procedure comprising random priming, nick translation or amplification of a sample of genomic nucleic acid to generate segments of target genomic nucleic acid followed by a step comprising fragmentation or enzymatic digestion of the segments to generate a sample of target genomic nucleic acid consisting of sizes smaller than about 200 bases. In other embodiments, the sample of target genomic nucleic acid is further prepared, e.g., fragmented, using procedures comprising mechanical fragmentation, e.g., shearing, or, enzymatic digestion, e.g., DNase enzyme, or equivalent, digestion, of a genomic nucleic acid (including the labeled nucleic acid generated by nick translation, random priming or amplification) to sizes smaller than about 200 bases, or, smaller than fragments of about 175 bases; about 150 bases; about 125 bases; about 100 bases; about 75 bases; about 50 bases; about 40 bases; about 30 bases; or about 25 bases. In another embodiment, the sample of target genomic nucleic acid (including the labeled target nucleic acid generated by nick translation, random priming or amplification) is prepared using a procedure comprising fragmentation of a genomic DNA to sizes smaller than about 200 bases by applying shearing forces sufficient to fragment genomic DNA followed by DNase or equivalent enzyme digestion of the sheared DNA to sizes smaller than about 200 bases, or, smaller than fragments of about 150 bases; about 125 bases; about 100 bases; about 75 bases; about 50 bases; about 40 bases; about 30 bases; or about 25 bases.

In this method, the conditions allowing hybridization of the target nucleic acid to the probe nucleic acid can comprise stringent hybridization conditions, or, alternatively, can also comprise stringent wash conditions. In alternative embodiments the stringent hybridization conditions can comprise a temperature of about 55° C. to about 60° C. to about 65° C. In other embodiments, the temperature of hybridization is changed at least once (or, many times) during the hybridization step. Also as described, below, the amount of humidity (i.e., water vapor) under which hybridization is performed can be modified at least once, or several times, during the hybridization step. The changes in temperature and/or humidity can be stepwise, or, gradual. The changes can continue throughout the hybridization procedure, or, any part of the hybridization step.

In one embodiment, the random priming, nick translation or amplification (using, e.g., degenerate primers) of the sample of genomic nucleic acid is used to generate segments of target genomic nucleic acid that incorporate detectably labeled base pairs into the segments. Alternatively, the incorporated base pairs can be modified or synthetic analog base pairs to allow attachment of detectable moieties to the base pairs. In one embodiment, the detectable label comprises a fluorescent dye, such as Cy3™ or Cy5™, or equivalent, a rhodamine, a fluorescein or an aryl-substituted 4,4-difluoro-4-bora-3a, 4a-diaza-s-indacene dye or equivalents.

In one embodiment, the target nucleic acid consists essentially of DNA derived from a human. The sample of target genomic nucleic acid can comprise sequences representing a defined fragment of a chromosome or substantially one or more entire chromosomes. The sample of target genomic nucleic acid can comprise sequences representing substantially an entire genome. In an alternative embodiment, the DNA from which the target or the probe nucleic acid is derived from a mammal, such as a mouse or a human genome.

The invention also provides a composition comprising a sample of target nucleic acid comprising fragments of genomic nucleic acid labeled with at least one detectable moiety, wherein each labeled fragment consists of a length smaller than about 200 bases, and the sample of labeled target genomic nucleic acid comprises sequences representing substantially a complete chromosome, or, substantially a complete genome. In alternative embodiments, the target genomic nucleic acid is smaller than about 175 bases, about 150 bases; about 125 bases; about 100 bases; about 75 bases; about 50 bases; about 40 bases; about 30 bases; or about 25 bases. In another embodiment, each labeled fragment consists of a length between about 30 bases and about 150 bases. In one embodiment, the target nucleic acid of the composition consists essentially of DNA derived from a human. The sample of target genomic nucleic acid can comprise sequences representing a defined fragment of a chromosome or substantially one or more entire chromosomes. The sample of target genomic nucleic acid can comprise sequences representing substantially an entire genome. In an alternative embodiment, the genome comprises a mammalian genome, such as a mouse or a human genome. In alternative embodiments, the composition can comprise any detectable label, e.g., it can comprises Cy3™ or Cy5™.

The invention also provides kits comprising a sample of target nucleic acid and printed matter, wherein the target nucleic acid comprises fragments of genomic nucleic acid labeled with a detectable moiety, wherein each labeled fragment consists of a length smaller than about 200 bases and the sample of labeled target genomic nucleic acid comprises sequences representing a defined part of or substantially an entire chromosome or genome; wherein the printed matter comprises instructions on hybridizing the sample of target nucleic acid to a nucleic acid array. In alternative embodiments, the kits' target genomic nucleic acid is smaller than about 175 bases, about 150 bases; about 125 bases; about 100 bases; about 75 bases; about 50 bases; about 40 bases; about 30 bases; or about 25 bases. In an alternative embodiment, the genomic DNA from which the target or the probe is derived comprises a mammalian genome, such as a mouse or a human genome.

The invention provides a method for hybridizing a sample of labeled nucleic acid targets to a plurality of nucleic acid probes, comprising the following steps: (a) providing a sample of nucleic acid targets comprising fluorescent-labeled nucleic acid fragments and a plurality of nucleic acid probes, wherein the fluorescent label is sensitive to Oxidation; (b) contacting the nucleic acid target and nucleic acid probe of step (a) under conditions allowing hybridization of the sample with the probe, wherein the hybridization conditions comprise use of a hybridization solution comprising at least one antioxidant, wherein the amount of antioxidant in the solution is sufficient to inhibit the oxidation of the fluorescent label under the hybridization conditions. In one embodiment, the fluorescent label comprises Cy5™ or equivalent. In alternative embodiments, the fluorescent dye comprises a rhodamine, a fluorescein or an aryl-substituted 4,4-difluoro-4-bora-3a, 4a-diaza s-indacene dye or equivalents.

The invention also provides a method for hybridizing a sample of Cy5™10 labeled nucleic acid targets to a plurality of nucleic acid probes, comprising the following steps: (a) providing a sample of nucleic acid targets comprising Cy5™-labeled nucleic acid fragments and a plurality of nucleic acid probes; (b) contacting the nucleic acid target and nucleic acid probe of step (a) under conditions allowing hybridization of the sample with the probe, wherein the hybridization conditions comprise use of a hybridization solution comprising at least one antioxidant, wherein the amount of antioxidant in the solution is sufficient to inhibit the oxidation of the Cy5™ under the hybridization conditions. The invention also provides a wash solution comprising a Cy5™-labeled nucleic acid comprising at least one antioxidant, wherein the amount of antioxidant in the solution is sufficient to inhibit the oxidation of the Cy5™ under the hybridization conditions.

The invention provides a composition comprising a sample of Cy5™-labeled nucleic acid in a solution comprising at least one antioxidant.

The invention also provides a kit comprising a sample of fluorescent-labeled nucleic acid in a solution comprising at least one antioxidant and printed matter, wherein the printed matter comprises instructions on using the labeled nucleic acid in a hybridization reaction with another nucleic acid. In alternative embodiments, the fluorescent dye comprises a rhodamine, a fluorescein or an aryl-substituted 4,4-difluoro-4-bora-3a, 4a-diaza s-indacene dye or equivalents. The invention also provides a kit comprising a sample of Cy5™-labeled nucleic acid in a solution comprising at least one antioxidant and printed matter, wherein the printed matter comprises instructions on using the Cy5™-labeled nucleic acid in a hybridization reaction with another nucleic acid. The kits can further comprise a wash solution, including a wash solution comprising at least one antioxidant.

In alternative embodiments, the antioxidant is present in solution, e.g., in a hybridization, wash and/or other solution, at a concentration of about 25 mM to about 1 M, about 50 mM to about 750 mM, about 50 mM to about 500 mM, and about 100 mM to about 500 mM.

In these compositions and methods, in alternative embodiments, the antioxidant comprises a mercapto-containing compound, or equivalent, such as a 2-mercaptoethylamine, a thiol N-acetylcysteine, an ovothiol, a 4-mercaptoimidazole. In another embodiment, the antioxidant comprises an antioxidant vitamin-containing compound, such as an ascorbic acid (Vitamin C) or a tocopherol (Vitamin E), or equivalent. In another embodiment, the antioxidant comprises a propyl gallate, such as an n-propyl gallate, or equivalent. In another embodiment, the antioxidant comprises a beta-carotene, or equivalent. In another embodiment, the antioxidant comprises a butylated hydroxytoluene (BHT) or a butylated hydroxyanisole (BHA), or equivalent.

The invention provides a method for hybridizing a sample of nucleic acid targets to a plurality of immobilized nucleic acid probes, comprising the following steps: (a) providing a sample of nucleic acid targets and a plurality of immobilized nucleic acid probes; (b) contacting the nucleic acid target and nucleic acid probe of step (a) under conditions allowing hybridization of the sample with the probe, wherein the hybridization conditions comprise a controlled hybridization environment comprising an unsaturated humidity environment. In alternative embodiments, the unsaturated humidity environment is controlled to about 90% humidity, about 80% humidity, about 70% humidity, about 60% humidity, about 50% humidity, about 40% humidity, about 30% humidity, and about 20% humidity.

In one embodiment, the humidity of the controlled environment is periodically changed during the hybridization of step (b). The change can be step-wise, or can be gradual. The humidity can be changed any number of times for any length of time. In alternative embodiments, the humidity is periodically changed at about three hour intervals, at about two hour intervals, at about one hour intervals, at about 30 minute intervals, at about 15 minute intervals or at about 5 minute intervals, or a combination thereof

In one embodiment, the hybridization conditions comprise a controlled temperature environment. The humidity of the controlled environment can be periodically changed during the hybridization of step (b). The change can be step-wise, or can be gradual. The temperature can be changed any number of times for any length of time. In alternative embodiments, the temperature is periodically changed at about three hour intervals, at about two hour intervals, at about one hour intervals, at about 30 minute intervals, at about 15 minute intervals or at about 5 minute intervals, or a combination thereof.

The invention provides a composition comprising an array of immobilized nucleic acids in a housing, wherein the housing comprises a component to measure and control the humidity in the housing. In one embodiment, the housing further comprises a component to measure and control the temperature in the housing. The housing can further comprise a component that allows programmable or preset control of the humidity and the temperature.

The invention provides an array of immobilized probe nucleic acids in a humidity-controlled housing, wherein the housing comprises a means to control the amount of humidity in the housing during hybridization of the probes to a target in an aqueous hybridization solution.

The invention provides an array of immobilized probe nucleic acids in a humidity-controlled housing, wherein the housing comprises a humidifier component that can control the amount of humidity in the housing during contact of the probes to an aqueous hybridization solution.

The invention provides a kit comprising an array of immobilized nucleic acids in a housing and printed matter, wherein the housing comprises a component to control the amount of humidity in the housing, a component to control the temperature in the housing, and a component to preset or program control of the humidity and the temperature, and the printed matter comprises instructions for presetting or programming conditions in the housing to hybridize a target to the immobilized nucleic acids of the array under controlled hybridization conditions that comprise fluctuation of humidity and temperature during a nucleic acid hybridization step.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits cited herein are hereby expressly incorporated by reference for all purposes.

### DESCRIPTION OF DRAWINGS

FIG. 1 is a schematic drawing of 5-amino-propargyl-2′-deoxycytidine 5′-triphosphate coupled to Cy5™ or Cy3™, as described in detail, below.

FIG. 2 is a schematic drawing of an unbalanced humidity hybridization format, as described in detail in Example 1, below.

Like reference symbols in the various drawings indicate like elements.

### DETAILED DESCRIPTION

The invention provides novel methods and compositions for array-based nucleic acid hybridizations. New methods and compositions are provided for generating a molecular profile of genomic DNA by hybridization of a target nucleic acid derived from genomic DNA to an immobilized nucleic acid probe, e.g., as in an “array-based comparative genomic hybridization (CGH).”

In one embodiment, the invention provides a method for generating a molecular profile of one or more genomes, or a defined portion of a genome, e.g., a chromosome or part of a chromosome, by hybridization of target nucleic acid derived from genomic DNA to an immobilized nucleic acid probe(s), e.g., in the form of an array. The method comprises contacting the immobilized nucleic acid segment (e.g., cloned DNA) with a sample of target nucleic acid comprising fragments of genomic nucleic acid labeled with a detectable moiety. Each labeled fragment consists of a length smaller than about 200 bases.

Use of labeled genomic DNA limited to this small size significantly improves the resolution of the molecular profile analysis, e.g., in array-based CGH. For example, use of such small fragments allows for significant suppression of repetitive sequences and other unwanted, “background” cross-hybridization on the immobilized nucleic acid. Suppression of repetitive sequence hybridization greatly increases the reliability of the detection of copy number differences (e.g., amplifications or deletions) or detection of unique sequences.

Labeled genomic DNA is a promiscuous mix containing more than 30% repetitive sequences and an unknown proportion of closely related sequences. Traditional protocols, particularly CGH methodologies, use significantly longer labeled genomic fragments than the fragments of the compositions and methods of the invention (fragments less than about 200 bases) to hybridize with immobilized genomic DNA, e.g., fixed metaphase chromosomes or nucleic acid arrays. These longer sequences cause a significant amount of unwanted cross-hybridization with repetitive and closely related sequences. In practicing the methods of the invention by using labeled target genomic nucleic acid smaller than about 200 bases effectively significantly reduces the amount of repetitive sequence hybridization and cross-hybridization from closely related sequences seen when traditional protocols are used. The resolution can also be significantly greater.

While the invention is not limited by any particular mechanism of action, the superior effectiveness of the methods of the invention may be because DNA probes fragmented to a smaller size (i.e., less than about 200 residues) have a lower possibility of partially hybridizing to closely related sequences under moderate or stringent hybridization conditions, e.g., the conditions typically used in array-based CGH. When the target sequence is sufficiently small, particularly under stringent hybridization conditions, only a perfectly matched sequence will hybridize at a specific hybridization temperature. For instance, in one exemplary scenario, two 200 base DNA molecules form a duplex molecule at 65° C. by pairing 100 bases; two 100 base single stranded dangling ends remain. These “dangling” single stranded ends can further hybridize to other DNA molecules. However, as the size of one or both of the molecules becomes less than 200 bases (with the hybridizing segments remaining 100 bases), the size of the “dangling end(s)” decreases and the probability that the non-hybridized ends will further hybridize to another fragment of DNA (resulting in “aggregating hybridization”) also proportionally decreases. In microarray hybridization, such as array-based CGH, this “aggregation hybridization” not only makes the hybridization less quantitative but also causes high background. Accordingly, the compositions and methods of the invention provide fragmented DNA probes to a size range of less than about 200 bases, e.g., between about 25 to about 30 to about 150 bases, or, about 50 to about 100 bases. In one embodiment, fragments of labeled nucleic acid derived from genomic DNA are first prepared by random priming, nick translation, amplification, or equivalents; followed by fragmentation to less than about 200 bases, as low as about 25 to about 30 bases; random priming, nick translation or amplification with degenerate primers typically generate labeled fragments ranging in size from about 200 to about 500 bases. Shear forces can be used to fragment this labeled nucleic acid; however, with shearing it is very difficult to fragment DNA to a size smaller than 200 bases. Accordingly, additional techniques, e.g., enzyme digestion, e.g., by DNase, or equivalent, is used to generate the smaller labeled pieces used as targets in the methods and compositions of the invention.

In addition to controlling the size of labeled genomic nucleic acid used to hybridize with the immobilized array DNA, the invention also provides compositions and methods for increasing the stability of nucleic acid-label conjugates that are sensitive to oxidation in solution, particularly, in hybridization solutions. Labels that are sensitive to oxidants, including free radicals, include many fluorescent dyes, particularly, Cy5™. Oxidation of the fluorescent dye quenches its ability to transmit a detectable signal; thus the presence of compositions or conditions that can oxidize a dye can significantly adversely effect the results of a hybridization reaction. This is particularly important if hybridization signals are to be detected and analyzed quantitatively. Accordingly, use of antioxidants and free-radical formation inhibitors in the compositions and methods of the invention can significantly increase the level of detectable signal from, e.g., a fluor; when very low or small amounts of fluor need to be detected, protection of even small amounts of fluor can be significant.

One current paradigm of comparative hybridization (CGH) is to use the fluorescent dyes Cy3™ and Cy5™ to differentially label nucleic acid fragments from two samples, e.g., nucleic acid generated from a control versus a test cell or tissue. Because of their superior spectral property and stability, Cy3™ and Cy5™ are almost exclusively used in current comparative hybridization protocols. Many commercial instruments are designed to accommodate to detection of these two dyes.

However, Cy5™ is not stable in most currently used hybridization solutions. Before this invention, loss of Cy5™ signal in the labeling reactions was mistakenly attributed to a low Cy5™ incorporation rate; incorporation of Cy5™-base conjugates into a nucleic acid fragment typically generated by primer extension of genomic DNA samples. While the invention is not limited by any particular mechanism of action, the present inventors found that the instability of Cy5™ at elevated temperature (e.g., at temperatures used for array-based CGH hybridization and other stringent hybridization procedures) is due to a long unsaturated carbon chain in its molecular backbone that is susceptible to radical attack. To increase the stability of Cy5™, or fluors or other oxidation-sensitive compounds, the invention provides methods and compositions that incorporate antioxidants and free radical scavengers in the hybridization mix, and, in one embodiment, the hybridization and the wash solutions. Using the methods and compositions of the invention, Cy5™ signals are dramatically increased and longer hybridization times are possible.

To further increase the hybridization sensitivity, the invention provides novel hybridization formats, or methodologies. In one embodiment of the invention, the hybridization is carried out in a controlled, unsaturated humidity environment (current methodologies/ protocols typically use 100% or near saturated humidity, see, e.g., Shalon (1996) Genome Res. 6:639-6450). In this embodiment of the invention, hybridization efficiency is significantly improved if the humidity is not saturated.

In another embodiment of the invention, the hybridization efficiency is further improved if the humidity is dynamically controlled, i.e., if the humidity changes during hybridization. Mass transfer will be facilitated in a dynamically balanced humidity environment. The humidity in the hybridization environment can be adjusted stepwise or continuously. Also provided are array devices comprising housings and controls that allow the operator to control the humidity during pre-hybridization, hybridization, wash and/or detection stages. In one embodiment, the device has detection, control and memory components to allow pre-programming of the humidity (and temperature (see below), and other parameters) during the entire procedural cycle, including pre-hybridization, hybridization, wash and detection steps.

The novel hybridization methods of the invention also provide hybridization conditions comprising temperature fluctuation. As is seen when the humidity is controllably changed, mass transfer is also facilitated in a dynamically balanced temperature environment. Hybridization has much better efficiency in a changing temperature environment as compared to conditions where the temperature is set precisely or at relatively constant level (e.g., plus or minus a couple of degrees, as with most commercial ovens). While the invention is not limited by any particular mechanism of action, the mixing caused either by temperature or humidity fluctuation increases hybridization efficiency. As noted above, the invention also provides devices for carrying out array-based hybridizations under precisely controlled environmental conditions, including dynamic control of temperature, humidity and other factors. Reaction chamber temperatures can be fluctuatingly modified by, e.g., an oven, or other device capable of creating changing temperatures.

The novel hybridization methods of the invention also provide hybridization conditions comprising osmotic fluctuation. Hybridization efficiency (i.e., time to equilibrium) can also be enhanced by a hybridization environment that comprises changing hyper-/hypo-tonicity, e.g., a solute gradient. In one embodiment, a solute gradient is created in the device. In one exemplary device, a low salt hybridization solution is placed on one side of the array hybridization chamber and a higher salt buffer is placed on the other side to generate a solute gradient in the chamber.

Definitions

Unless defined otherwise, all technical and scientific terms used herein have the meaning commonly understood by a person skilled in the art to which this invention belongs. As used herein, the following terms have the meanings ascribed to them unless specified otherwise.

The term “antioxidant” includes any compound capable of inhibiting or preventing the oxidation of a second compound, such as a fluorescent dye, and, in particular, the fluorochrome Cy5™ in an aqueous solution. Accordingly, the term also includes all compounds which exhibit an anti-free radical protective effect. Antioxidants and free radical scavengers are described in detail, below. A compound is considered to be an effective anti-oxidant or free-radical inhibitor if it has any degree of protective effect on the oxidation-sensitive compound during hybridization (i.e., less Cy5™ fluor oxidized during the course of the hybridization procedure).

The term “aryl-substituted 4,4-difluoro-4-bora-3a, 4a-diaza-s-indacene dye” as used herein includes all “boron dipyrromethene difluoride fluorophore” or “BODIPY” dyes and “dipyrrometheneboron difluoride dyes” (see, e.g., U.S. Pat. No. 4,774,339), or equivalents, are a class of fluorescent dyes commonly used to label nucleic acids for their detection when used in hybridization reactions; see, e.g., Chen (2000) J. Org Chem. 65:2900-2906: Chen (2000) J. Biochem. Biophys. Methods 42:137-151. See also U.S. Pat. Nos. 6,060,324; 5,994,063; 5,614,386; 5,248,782; 5,227,487; 5,187,288.

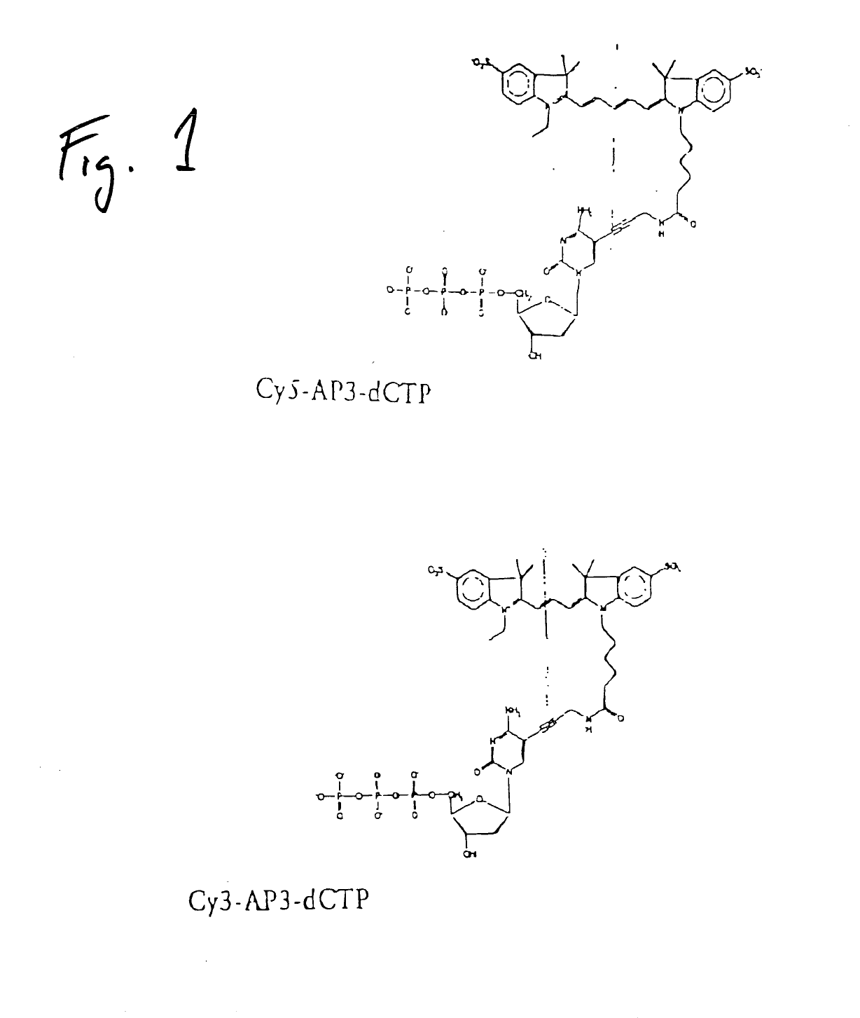

The terms “cyanine 5” or “Cy5™” and “cyanine 3” or “Cy3™” refer to fluorescent cyanine dyes produced by Amersham Pharmacia Biotech (Piscataway, N.J.) (Amersham Life Sciences, Arlington Heights, Ill.), as described in detail, below, or equivalents. See U.S. Pat. Nos. 6,027,709; 5,714,386; 5,268,486; 5,151,507; 5,047,519. These dyes are typically incorporated into nucleic acids in the form of 5-amino-propargyl-2′-deoxycytidine 5′-triphosphate coupled to Cy5™ or Cy3™. See FIG. 1.

The term “fluorescent dye” as used herein includes all known fluors, including rhodamine dyes (e.g., tetramethylrhodamine, dibenzorhodamine, see, e.g., U.S. Pat. No. 6,051,719); fluorescein dyes; “BODIPY” dyes and equivalents (e.g., dipyrrometheneboron difluoride dyes, see, e.g., U.S. Pat. No. 5,274,113); derivatives of 1-[isoindolyl]methylene-isoindole (see, e.g., U.S. Pat. No. 5,433,896); and all equivalents. See also U.S. Pat. Nos. 6,028,190; 5,188,934.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be used to identify and isolate nucleic acids within the scope of the invention. Those of ordinary skill will readily recognize that alternative but comparable hybridization and wash conditions can be utilized to provide conditions of similar stringency. However, the selection of a hybridization format is not critical, as is known in the art, it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equilvalent hybridization and wash conditions and for reagents and buffers, e.g., SSC buffers and equivalent reagents and conditions.

The term “labeled with a detectable composition” or “labeled with a detectable moiety” as used herein refers to a nucleic acid attached to a detectable composition, i.e., a label, as described in detail, below. This includes incorporation of labeled bases (or, bases which can bind to a detectable label) into the nucleic acid by, e.g., nick translation, random primer extension, amplification with degenerate primers, and the like. The label can be detectable by any means, e.g., visual, spectroscopic, photochemical, biochemical, immunochemical, physical or chemical means.

The term “a molecular profile of genomic DNA” means detection of regions of amplification, deletions and/ or unique sequences in a test sample of nucleic acid representing a genomic DNA as compared to a control (e.g., “normal”) sample of DNA.

The term “nucleic acid” as used herein refers to a deoxyribonucleotide or ribonucleotide in either single-or double-stranded form. The term encompasses nucleic acids containing known analogues of natural nucleotides. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,296; Samstag (1996) Antisense Nucleic Acid Drug Dev 6:153-156). The term nucleic acid is used interchangeably with gene, DNA, RNA, cDNA, mRNA, oligonucleotide primer, probe and amplification product.

The terms “array” or “microarray” or “DNA array” or “nucleic acid array” or “biochip” as used herein is a plurality of target elements, each target element comprising a defined amount of one or more nucleic acid molecules, or probes (defined below), immobilized a solid surface for hybridization to sample nucleic acids, as described in detail, below. The term “probe(s)” or “nucleic acid probe(s)” as used herein, is defined to be a collection of one or more nucleic acid fragments (e.g., immobilized nucleic acid, e.g., a nucleic acid array) whose hybridization to a sample of target nucleic acid (defined below) can be detected.

The term “sample of nucleic acid targets” or “sample of nucleic acid” as used herein refers to a sample comprising DNA or RNA, or nucleic acid representative of DNA or RNA isolated from a natural source, in a form suitable for hybridization (e.g., as a soluble aqueous solution) to another nucleic acid or polypeptide or combination thereof (e.g., immobilized probes). The nucleic acid may be isolated, cloned or amplified; it may be, e.g., genomic DNA, mRNA, or cDNA from substantially an entire genome, substantially all or part of a particular chromosome, or selected sequences (e.g. particular promoters, genes, amplification or restriction fragments, cDNA, etc.). The nucleic acid sample may be extracted from particular cells or tissues. The cell or tissue sample from which the nucleic acid sample is prepared is typically taken from a patient suspected of having a genetic defect or a genetically-linked pathology or condition, e.g., a cancer, associated with genomic nucleic acid base substitutions, amplifications, deletions and/or translocations. Methods of isolating cell and tissue samples are well known to those of skill in the art and include, but are not limited to, aspirations, tissue sections, needle biopsies, and the like. Frequently the sample will be a “clinical sample” which is a sample derived from a patient, including sections of tissues such as frozen sections or paraffin sections taken for histological purposes. The sample can also be derived from supernatants (of cells) or the cells themselves from cell cultures, cells from tissue culture and other media in which it may be desirable to detect chromosomal abnormalities or determine amplicon copy number. In some cases, the nucleic acids may be amplified using standard techniques such as PCR, prior to the hybridization. In alternative embodiments, the target nucleic acid may be unlabeled, or labeled (as, e.g., described herein) so that its binding to the probe (e.g., oligonucleotide, or clone, immobilized on an array) can be detected. The probe an be produced from and collectively can be representative of a source of nucleic acids from one or more particular (pre-selected) portions of, e.g., a collection of polymerase chain reaction (PCR) amplification products, substantially an entire chromosome or a chromosome fragment, or substantially an entire genome, e.g., as a collection of clones, e.g., BACs, PACs, YACs, and the like (see below). The probe or genomic nucleic acid sample may be processed in some manner, e.g., by blocking or removal of repetitive nucleic acids or by enrichment with selected nucleic acids.

Generating and Manipulating Nucleic Acids

The invention provides compositions, including nucleic acid arrays, and methods for performing nucleic acid hybridization reactions. As described herein, the labeled target nucleic acid for analysis and the immobilized nucleic acid on the array can be representative of genomic DNA, including defined parts of, or entire, chromosomes, or entire genomes. In several embodiments, the arrays and methods of the invention are used in comparative genomic hybridization (CGH) reactions, including CGH reactions on arrays (see, e.g., U.S. Pat. Nos. 5,830,645; 5,976,790). These reactions compare the genetic composition of test versus controls samples; e.g., whether a test sample of genomic DNA (e.g., from a cell suspected of having a genetic defect) has amplified or deleted or mutated segments, as compared to a “negative” control, e.g., “normal” wild type genotype, or “positive” control, e.g., known cancer cell or cell with a known defect, e.g., a translocation or amplification or the like.

In other embodiments, the test sample comprises fragments of nucleic acid representative of defined parts of a chromosome or genome, or the entire genome. The test sample can be labeled, e.g., with a detectable moiety, e.g., a fluorescent dye. Typically, the test sample nucleic acid is labeled with a fluor and the control (e.g., “normal”) sample is labeled with a second dye (e.g., Cy3™ and Cy5™). Test and control samples are both applied to the immobilized probes (e.g., on the array) and, after hybridization and washing, the location (e.g., spots on the array) and amount of each dye are read. The immobilized nucleic acid can be representative of any part of or all of a chromosome or genome. If immobilized to an array, this nucleic acid can be in the form of cloned DNA, e.g., YACs, BACs, PACs, and the like, as described herein. As is typical of array technology, each “spot” on the array has a known sequence, e.g., a known segment of genome or other sequence. The invention can be practiced in conjunction with any method or protocol or device known in the art, which are well described in the scientific and patent literature.

General Techniques

The nucleic acids used to practice this invention, whether RNA, cDNA, genomic DNA, vectors, viruses or hybrids thereof, may be isolated from a variety of sources, genetically engineered, amplified, and/or expressed/ generated recombinantly. Any recombinant expression system can be used, including, in addition to bacterial cells, e.g., mammalian, yeast, insect or plant cell expression systems.

Alternatively, these nucleic acids can be synthesized in vitro by well-known chemical synthesis techniques, as described in, e.g., Carruthers (1982) Cold Spring Harbor Symp. Quant. Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Double stranded DNA fragments may then be obtained either by synthesizing the complementary strand and annealing the strands together under appropriate conditions, or by adding the complementary strand using DNA polymerase with a primer sequence.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used in the compositions and methods of the invention is to clone from genomic samples, and, if necessary, screen and re-clone inserts isolated (or amplified) from, e.g., genomic clones or cDNA clones or other sources of complete genomic DNA. Thus, forms of genomic nucleic acid used in the methods and compositions of the invention (including arrays and test samples) include genomic or cDNA libraries contained in, or comprised entirely of, e.g., mammalian artificial chromosomes (see, e.g., Ascenzioni (1997) Cancer Lett. 118:135-142; U.S. Pat. Nos. 5,721,118; 6,025,155) (including human artificial chromosomes, see, e.g., Warburton (1997) Nature 386:553-555; Roush (1997) Science 276:38-39; Rosenfeld (1997) Nat. Genet. 15:333-335); yeast artificial chromosomes (YAC); bacterial artificial chromosomes (BAC); P1 artificial chromosomes (see, e.g., Woon (1998) Genomics 50:306316; Boren (1996) Genome Res. 6:1123-1130); PACs (a bacteriophage P1-derived vector, see, e.g., Ioannou (1994) Nature Genet. 6:84-89; Reid (1997) Genomics 43:366-375; Nothwang (1997) Genomics 41:370-378; Kern (1997) Biotechniques 23:120-124); cosmids, plasmids or cDNAs. BACs are vectors that can contain 120 Kb or greater inserts. BACs are based on the E. coli F factor plasmid system and simple to manipulate and purify in microgram quantities. Because BAC plasmids are kept at one to two copies per cell, the problems of rearrangement observed with YACs, which can also be employed in the present methods, are eliminated; see, e.g., Asakawa (1997) Gene 69-79; Cao (1999) Genome Res. 9:763-774. BAC vectors can include marker genes, such as, e.g., luciferase and green fluorescent protein genes (see, e.g., Baker (1997) Nucleic Acids Res 25:1950-1956). YACS can also be used and contain inserts ranging in size from 80 to 700 kb, see, e.g., Tucker (1997) Gene 199:25-30; Adam (1997) Plant J. 11:1349-1358; Zeschnigk (1999) Nucleic Acids Res. 27:21. P1 is a bacteriophage that infects E. coli that can contain 75-100 Kb DNA inserts (see, e.g., Mejia (1997) Genome Res 7:179-186; Ioannou (1994) Nat Genet 6:84-89), and are screened in much the same way as lambda libraries. See also Ashworth (1995) Analytical Biochem. 224:564-571; Gingrich (1996) Genomics 32:65-74. Sequences, inserts, clones, vectors and the like can be isolated from natural sources, obtained from such sources as ATCC or GenBank libraries or commercial sources, or prepared by synthetic or recombinant methods.

Amplification of Nucleic Acids

Amplification using oligonucleotide primers can be used to generate nucleic acids used in the compositions and methods of the invention, to detect or measure levels of test or control samples hybridized to an array, and the like. Amplification, typically with degenerate primers, is also useful for incorporating detectable probes (e.g., Cy5™-or Cy3™-cytosine conjugates) into nucleic acids representative of test or control genomic DNA to be used to hybridize to immobilized genomic DNA. The skilled artisan can select and design suitable oligonucleotide amplification primers. Amplification methods are also well known in the art, and include, e.g., polymerase chain reaction, PCR (PCR PROTOCOLS, A GUIDE TO METHODS AND APPLICATIONS, ed. Innis, Academic Press, New York (1990) and PCR STRATEGIES (1995), ed. Innis, Academic Press, Inc., New York, ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564. See, e.g., U.S. Pat. No. 6,063,571, describing use of polyamide-nucleic acid derivatives (PNAs) in amplification primers.

Hybridizing Nucleic Acids

In practicing the methods of the invention and using the compositions of the invention, test and control samples of nucleic acid are hybridized to immobilized probe nucleic acid, e.g., on arrays. In one embodiment, the hybridization and/or wash conditions are carried out under moderate to stringent conditions. An extensive guide to the hybridization of nucleic acids is found in, e.g., Sambrook Ausubel, Tijssen. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or a filter in a Southern or northern blot is 42° C. using standard hybridization solutions (see, e.g., Sambrook), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook). Often, a high stringency wash is preceded by a medium or low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 4° C. for 15 minutes.

Detectably Labeled Nucleic Acids

In some embodiments, the methods and compositions of the invention use nucleic acids representative of genomic DNA that have been conjugated to a detectable moiety, or into a nucleoside base conjugated to a detectable moiety (e.g., Cy3™ or Cy5™) has been incorporated (or, alternatively, a moiety that itself can bind to a detectable composition). The test samples can comprise labeled fragments of nucleic acid representative of part of or all of a chromosome, or an entire genome. In one embodiment, the test sample nucleic acid is conjugated with one label and the control sample is conjugated with a second label, wherein each label is differentially detectable (e.g., emits a difference signal). Test and control samples are both applied to the immobilized probes (e.g., on the array) and, after hybridization and washing, the location (e.g., spots on the array) and amount of each label are read simultaneously or sequentially.

Useful labels include ³²p, ³⁵S, ³H, ¹⁴C, ¹²⁵I, ¹³¹I; fluorescent dyes (e.g., Cy5™, Cy3™, FITC, rhodamine, lanthanide phosphors, Texas red), electron-dense reagents (e.g. gold), enzymes, e.g., as commonly used in an ELISA (e.g., horseradish peroxidase, betagalactosidase, luciferase, alkaline phosphatase), colorimetric labels (e.g. colloidal gold), magnetic labels (e.g. Dynabeads™), biotin, dioxigenin, or haptens and proteins for which antisera or monoclonal antibodies are available. The label can be directly incorporated into the nucleic acid or other target compound to be detected, or it can be attached to a probe or antibody which hybridizes or binds to the target. A peptide can be made detectable by incorporating (e.g., into a nucleoside base) predetermined polypeptide epitopes recognized by a secondary reporter (e.g., leucine zipper pair sequences, binding sites for secondary antibodies, transcriptional activator polypeptide, metal binding domains, epitope tags). Label can be attached by spacer arms of various lengths to reduce potential steric hindrance or impact on other useful or desired properties. See, e.g., Mansfield (1995) Mol Cell Probes 9:145-156. In array-based CGH, typically fluors are paired together (one labeling control and another the test nucleic acid), e.g., rhodamine and fluorescein (see, e.g., DeRisi (1996) Nature Genetics 14:458-460), or lissamine-conjugated nucleic acid analogs and fluorescein-conjugated nucleotide analogs (see, e.g., Shalon (1996) supra); or Spectrum Red™ and Spectrum Green™ (Vysis, Downers Grove, Ill.) or Cy3™ and Cy5™ (see below).

Cyanine and related dyes, such as merocyanine, styryl and oxonol dyes, are particularly strongly light-absorbing and highly luminescent, see, e.g., U.S. Pat. Nos. 4,337,063; 4,404,289; 6,048,982. In one embodiment, Cy3™ and Cy5™ are used together; both are fluorescent cyanine dyes produced by Amersham Life Sciences (Arlington Heights, Ill.). They can be incorporated into “target” nucleic acid by transcription (e.g., by random-primer labeling using Klenow polymerase, or “nick translation,” or, amplification, or equivalent) of samples of genomic DNA, wherein the reaction incorporates Cy3™-or Cy5™-dCTP conjugates mixed with unlabeled dCTP. According to manufacturer's instructions, if generating labeled target by PCR, a mixture of 33% modified to 66% unmodified dCTP gives maximal incorporation of label; when modified dCTP made up 50% or greater, the PCR reaction was inhibited. Cy5™ is typically excited by the 633 nm line of HeNe laser, and emission is collected at 680 nm. See also, e.g., Bartosiewicz (2000) Archives of Biochem. Biophysics 376:66-73; Schena (1996) Proc. Natl. Acad. Sci. USA 93:10614-10619; Pinkel (1998) Nature Genetics 20:207-211; Pollack (1999) Nature Genetics 23:41-46.

Methods for labeling nucleic acids with fluorescent dyes and for the simultaneous detection of multiple fluorophores are known in the art, see, e.g., U.S. Pat. Nos. 5,539,517; 6,049,380; 6,054,279; 6,055,325. For example a spectrograph can image an emission spectrum onto a two-dimensional array of light detectors; a full spectrally resolved image of the array is thus obtained. Photophysics of the fluorophore, e.g., fluorescence quantum yield and photodestruction yield, and the sensitivity of the detector are read time parameters for an oligonucleotide array. With sufficient laser power and use of Cy5™ and/or Cy3™, which have lower photodestruction yields an array can be read in less than 5 seconds.

When using two fluors together (e.g., as in a CGH), such as Cy3™ and Cy5™, it is necessary to create a composite image of both fluors. To acquire the two images, the array can be scanned either simultaneously or sequentially. Charge-coupled devices, or CCDs, are commonly used in microarray scanning systems.

Data analysis can include the steps of determining, e.g., fluorescent intensity as a function of substrate position, removing “outliers” (data deviating from a predetermined statistical distribution), or calculating the relative binding affinity of the targets from the remaining data. The resulting data can be displayed as an image with color in each region varying according to the light emission or binding affinity between targets and probes. See, e.g., U.S. Pat. Nos. 5,324,633; 5,863,504; 6,045,996. The invention can also incorporate a device for detecting a labeled marker on a sample located on a support, see, e.g., U.S. Pat. No. 5,578,832.

Fragmentation and Digestion of Labeled Genomic Nucleic Acid

The invention provides methods and compositions using labeled genomic fragments of less than about 200 bases to as small as about 25 to about 30 bases. Typical CGH protocols use considerably larger labeled nucleic acids. In fact, some protocols recommend use of long fragments to improve intensity and uniformity of hybridization (See, e.g., Kalloniemi (1994) Genes, Chromosomes & Cancer 10:231-243).

As discussed above, as the size of target labeled nucleic acids becomes less than 200 bases, the size of unhybridized “dangling ends” decreases and the probability that the non-hybridized ends will further hybridize to another fragment of DNA, resulting in “aggregating hybridization,” also decreases. In microarray hybridization, such as array-based CGH, this “aggregation hybridization” not only makes the hybridization less quantitative but also causes high background. Accordingly, the compositions and methods of the invention provide fragmented DNA probes to a size range of less than about 200 bases, as low as about 30 bases.

Typically, the labeled nucleic acid used in the hybridization procedures is generated from genomic DNA by standard “random priming,” “nick translation” or degenerate PCR amplification (see, e.g., Sambrook, Ausubel; Speicher (1993) Hum. Mol. Genet. 2:1907-1914). However, the resultant fragments average about 200 to 400 bases, or more (see, e.g., Heiskanen (2000) Cancer Res. 60:799-802, where total genomic DNA labeled with biotin by nick translation generated fragment sizes of between 400 and 2000 bases). The fragment length can be modified by adjusting the ratio of DNase to DNA polymerase in the nick translation reaction; standard nick translation kits typically generate 300 to 600 base pair fragments (See, e.g., Kalloniemi (1994) supra).

To further fragment the labeled nucleic acid to segments below 200 bases, down to as low as about 25 to 30 bases, random enzymatic digestion of the DNA is carried out, using, e.g., a DNA endonucleases, e.g., DNase (see, e.g., Herrera (1994) J. Mol. Biol. 236:405-411; Suck (1994) J. Mol. Recognit. 7:65-70), or, the two-base restriction endonuclease CviJI (see, e.g., Fitzgerald (1992) Nucleic Acids Res. 20:3753-3762) and standard protocols, see, e.g., Sambrook, Ausubel, with or without other fragmentation procedures.

Other procedures can also be used to fragment genomic DNA, e.g. mechanical shearing, sonication (see, e.g., Deininger (1983) Anal. Biochem. 129:216-223), and the like (see, e.g., Sambrook, Ausubel, Tijssen). For example, one mechanical technique is based on point-sink hydrodynamics that result when a DNA sample is forced through a small hole by a syringe pump, see, e.g., Thorstenson (1998) Genome Res. 8:848-855. See also, Oefner (1996) Nucleic Acids Res. 24:3879-3886; Ordahl (1976) Nucleic Acids Res. 3:2985-2999. Fragment size can be evaluated by a variety of techniques, including, e.g., sizing electrophoresis, as by Siles (1997) J. Chromatogr. A. 771:319-329, that analyzed DNA fragmentation using a dynamic size-sieving polymer solution in a capillary electrophoresis.

Fragment sizes can also be determined by, e.g., matrix-assisted laser desorption/ionization time-of-flight mass spectrometry, see, e.g., Chiu (2000) Nucleic Acids Res. 28:E31.

Antioxidant and Free Radical Scavengers

The invention provides methods and compositions comprising antioxidants and free radical scavengers, many of which are known in the art. For example, in one embodiment, the antioxidant can comprise a mercapto-containing compound, or equivalent, such as a 2-mercapto-ethylamine, a thiol N-acetylcysteine, an ovothiol, a 4-mercaptoimidazole. A vitamin-containing compound, such as an ascorbic acid (Vitamin C) or a tocopherol (Vitamin E), or equivalent, can also be used. Tocopherols can include variations and derivative forms, e.g., alpha-D-tocopherol, alpha-DL-tocopherol, alpha.-D-tocopherol acetate, alpha-DL-tocopherol acetate, or alpha-D-tocopherol acid succinate (see, e.g., U.S. Pat. Nos. 6,048,891; 6,048,988; 6,056,897). In another embodiment, the antioxidant comprises a propyl gallate, such as an n-propyl gallate, or equivalent. Betacarotenes, or equivalent, or butylated hydroxytoluene (BHT) or butylated hydroxyanisole (BHA), or equivalent, can also be used.

Peptide and peptide derivatives have also been described to have antioxidant activity, see, e.g., U.S. Pat. No. 5,804,555, describing the antioxidant action of a hydrolysate of lactoferrins. 2-mercaptoimidazole or 4-mercaptohistidine derivatives have also been described to have antioxidant activity, see, e.g., U.S. Pat. No. 6,056,965 and U.S. Pat. No. 4,898,878, respectively. Some cyclical hydroxylamines are useful for scavenging oxygen-centered free radicals, see, e.g., U.S. Pat. No. 5,981,548. Ascorbic acid 6-palmitate, dihydrolipoic acid have also been described as antioxidants, see, e.g., U.S. Pat. No. 5,637,315. See also U.S. Pat. No. 5,162,366.

Hybridization and wash solutions used in CGH and arrays are known in the art, see, e.g., Cheung (1999) Nature Genetics Supp. 21:15-19; see also, definitions discussion, above. The concentration of antioxidant in those solutions depends on a variety of factors: e.g., the composition of the hybridization or wash buffer; the concentration of composition to be “protected” from oxidation (e.g., Cy5™), the hybridization and wash conditions (e.g., length of time, heat, humidity, etc.). Thus, in various embodiments, the amount of antioxidant in a hybridization, wash or other solution, can be, e.g., at a concentration of about 25 mM to about 1 M, about 50 mM to about 750 mM, about 50 mM to about 500 mM, and about 100 mM to about 500 mM. However, any appropriate concentration of antioxidant or free radical scavenger can be used to practice the invention.

Additional effective antioxidants and free radicals can be readily determined, e.g., the development of a simple method for rapid screening of antioxidants in the preformulation phase of drug development is described by, e.g., Ugwu (1999) PDA J. Pharm. Sci. Technol. 53:252-259. Using an easily oxidizable drug substance containing a tetrahydroisoquinoline nucleus, the relative antioxidant efficacies can be determined by simultaneous measurement of dissolved oxygen depletion and drug disappearance rates in presence and absence of antioxidants. See also, e.g., Methods Enzymol. 1990; 186:1-766; U.S. Pat. No. 6,031,008.

Arrays, or “BioChips”

The invention provides improved variations of “arrays” or “microarrays” or “DNA arrays” or “nucleic acid arrays” or “biochips” (e.g., GeneChips®, Affymetrix, Santa Clara, Calif.). The arrays of the invention comprise housings comprising components for controlling humidity and temperature during the hybridization and wash reactions.

Arrays are generically a plurality of target elements, each target element comprising a defined amount of one or more nucleic acid molecules, or probes, immobilized a solid surface for hybridization to sample nucleic acids. The immobilized nucleic acids can contain sequences from specific messages (e.g., as cDNA libraries) or genes (e.g., genomic libraries), including, e.g., substantially all or a subsection of a chromosome or substantially all of a genome, including a human genome. Other target elements can contain reference sequences and the like. The target elements of the arrays may be arranged on the solid surface at different sizes and different densities. The target element densities will depend upon a number of factors, such as the nature of the label, the solid support, and the like. Each target element may comprise substantially the same nucleic acid sequences, or, a mixture of nucleic acids of different lengths and/or sequences. Thus, for example, a target element may contain more than one copy of a cloned piece of DNA, and each copy may be broken into fragments of different lengths, as described herein. The length and complexity of the nucleic acid fixed onto the target element is not critical to the invention. The array can comprise nucleic acids immobilized on a solid surface (e.g., nitrocellulose, glass, quartz, fused silica, plastics and the like). See, e.g., U.S. Pat. No. 6,063,338 describing multi-well platforms comprising cycloolefin polymers if fluorescence is to be measured. In some embodiments, the methods of the invention can be practiced on arrays of nucleic acids as described, for instance, in U.S. Pat. Nos. 6,045,996; 6,022,963; 6,013,440; 5,959,098; 5,856,174; 5,770,456; 5,556,752; 5,143,854; see also, e.g., WO 99/51773; WO 99/09217; WO 97/46313; WO 96/17958; see also, e.g., Johnston (1998) Curr. Biol. 8:R171-R174; Schummer (1997) Biotechniques 23:.1087-1092; Kern (1997) Biotechniques 23:120-124; Solinas-Toldo (1997) Genes, Chromosomes & Cancer 20:399-407; Bowtell (1999) Nature Genetics Supp. 21:25-32; Epstein (2000) Current Opinion in Biotech. 11:36-41.

Control of Humidity and Temperature During Hybridization

The invention provides methods and compositions where hybridization conditions comprise a controlled hybridization environment, particularly, an unsaturated humidity environment. The humidity and temperature of the controlled environment can be constant or periodically changed during the hybridization of step. The change can be stepwise, or can be gradual. In alternative embodiments, the unsaturated humidity environment is controlled to about 90% humidity, about 80% humidity, about 70% humidity, about 60% humidity, about 50% humidity, about 40% humidity, about 30% humidity, and about 20% humidity. In alternative embodiments, the humidity and/or temperature are periodically changed at about three hour intervals, at about two hour intervals, at about one hour intervals, at about 30 minute intervals, at about 15 minute intervals or at about 5 minute intervals, or a combination thereof.

The invention also provides an array of immobilized probe nucleic acids in a humidity-and/or temperature-controlled housing. In one embodiment, the housing comprises a component to measure and control the amount of humidity and/or the temperature in the housing during hybridization. For example, the devices of the invention can comprise any temperature detection or control component, which are known in the art, e.g., thermal control modules comprising Peltier heat transfer devices for the control of temperature (these can be incorporated into the housing), see, e.g., U.S. Pat. No. 6,017,434, using such devices in an electrophoretic medium; or the devices of the invention can comprise a sealed thermostatically controlled chamber in which fluids can easily be introduced (see, e.g., U.S. Pat. No. 5,945,334); or they can comprise a system for the temperature adjustment treatment of liquids (see, e.g., U.S. Pat. No. 5,919,622); or a reaction chamber for conducting elevated temperature reactions in a fluid-tight manner (see, e.g., U.S. Pat. No. 5,882,903); or a biological chip plate with a fluid handling device (see. e.g., U.S. Pat. No. 5,874,219); or a reaction vessel with a temperature control device manner (see, e.g., U.S. Pat. No. 5,460,780). The devices of the invention also can comprise any humidity or water vapor detection or control component, or an adaptation or variation thereof; many of such devices are known in the art, e.g., U.S. Pat. Nos. 4,436,674; 4,618,462; 4,921,642; 5,620,503; 5,806,762; and, 6,064,059, describing a device for detecting moisture conditions on a glass surface.

The component can include memory components to allow for pre-programming of hybridization conditions, including humidity and temperature and other environmental parameters.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The following example is offered to illustrate, but not to limit the claimed invention.

### Example 1

### Array-based Nucleic Acid Hybridization

The following example demonstrates that the methods of the invention provide an improved and efficient means to practice array-based CGH.

Making BAC Microarrays:

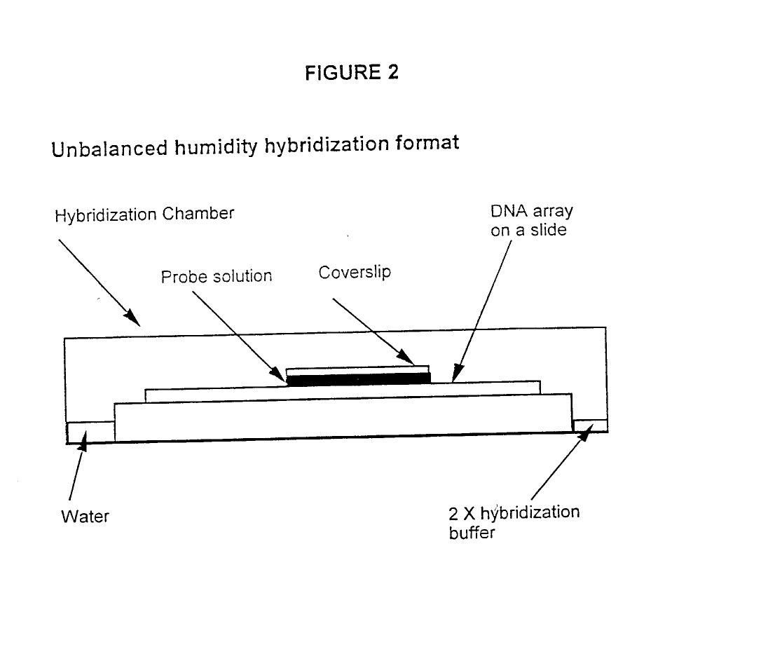

BAC clones greater than fifty kilobases (50 kb), and up to about 300 kb, were grown up in Terrific Broth medium (larger inserts, e.g., clones >300 kb, or smaller inserts, about 1 to 20 kb, can also be used). DNA was prepared by a modified alkaline lysis protocol (see, e.g., Sambrook). The DNA was chemically modified as described by U.S. Pat. No. 6,048,695. The modified DNA was then dissolved in proper buffer and printed directly on clean glass surfaces as described by U.S. Pat. No. 6,048,695. Usually multiple spots were printed for each clone. FIG. 2 is a schematic drawing of an unbalanced humidity hybridization format used in these studies.

Probe Labeling and DNase Enzyme Fragmentation:

A standard random priming method was used to label genomic DNA, see, e.g., Sambrook. Cy3™ or Cy5™ labeled nucleotides were supplemented together with corresponding unlabeled nucleotides at a molar ratio ranging from 0.0 to about 6 (unlabeled nucleotide to labeled nucleotides). Labeling was carried out at 37° C. for 2 to 10 hours. After labeling the reaction mix was heated up to 95° C. to 100° C. for 3 to 5 minutes to inactivate the polymerase and denature the newly generated, labeled “probe” nucleic acid from the template.

The heated sample was then chilled on ice for 5 minutes. “Calibrated” DNase (DNA endonuclease) enzyme was added to fragment the labeled template (generated by random priming). “Trace” amounts of DNase was added (final concentration was 0.2 to 2 ng/ml; incubation time 15 to 30 minutes) to digest/fragment the labeled nucleic acid to segments of about 30 to about 100 bases in size.

Blocking Repetitive Sequences Using Cot I DNA.

Cot I DNA was fragmented to sizes of between about 40 to 150 bases. 2 to 20 μg of fragmented Cot I DNA, together with about 10 to 30 μg of sheared salmon sperm or testes DNA (size range about 0.1 to 2 kb) (carrier DNA, also can be other unrelated DNA), was dissolved in 2× to 6×SSPE with 0.2% to 10% base hybridization buffer (see e.g., Sambrook). The mix was applied to the array area, which was subsequently covered with a coverslip. The array was placed in a humidified chamber at 60° C. for 2 to 16 hours

Hybridization of Labeled Probes to Arrays.

Two genomic fragments, each with 10,000 to 1,000,000 genome equivalence (derived from both human and mouse genomic DNA), were each labeled using either Cy3™ or Cy5™ fluorescent label, as described above. They were then co-precipitated with about 2 to 20 μg of Cot I and about 10 to 30 μg of carried DNA or about 50 to 100 μg yeast tRNA. The mixture was dissolved in 10 to 20 μl base hybridization buffer (see above).

Antioxidants Added to Hybridization Buffer

The antioxidant dithiothreitol (DTT) was added to a concentration of 10 to 500 mM to stabilize the fluorescent dyes. Other usable antioxidants include, e.g., n-propyl gallate, ascorbic acid (Vitamin C), Vitamin E (tocopherol), 2-mercaptoethylamine or other mercapto-containing compounds, as discussed above. The mix was applied to the array area, which was subsequently covered with a coverslip (see FIG. 2). Hybridization was carried out in a humidified chamber with an average humidity of about 90 to 95% at 60° C. overnight in an oven with approximately +/−3° C. of temperature fluctuation (temperature variation itself may cause fluctuations in the humidity in the closed clamber).

Humidity Conditions Fluctuated

Experiments also demonstrated that an unbalanced humidity environment significantly decreased the amount of time needed to reach equilibrium between soluble labeled nucleic acid and immobilized probe. A schematic of the device used in these experiments is presented in FIG. 2. Both “unsealed” coverslips (to provide a “dynamic” humidity condition) and sealed coverslips (to provide the control 100% humidity environment) were used.

If the coverslip was sealed to prevent exchange of water vapor (i.e., a dynamic humidity environment) the rate of hybridization was significantly worse (a significantly longer period of time was needed to reach equilibrium). Rate of hybridization was determined by measuring the amount of Cy3™ or Cy5™ -generated fluorescence, i.e., the amount of labeled nucleic acid, hybridized to the immobilized probes on the array; fluorescence was measured using standard devices, as described above.

Post-hybridization Washes:

The array was rinsed with high purity water several times after the coverslip was removed. The array was then washed in a solution comprising 0.1 to 2×SSC with 0.1 to 1% SDS and 5 to 10 mM DTT antioxidant for 30 to 60 minutes. The array was then rinsed extensively with high purity water at room temperature (RT).

Image Acquisition and Data Processing:

The fluorescent signals on microarrays are scanned into image files (a two color laser confocal scanner from GSI Lumonics (Oxnard, Calif.). For each array two images are acquired (for Cy3™ and Cy5™). The relative fluorescent level or fluorescent ratio, which represents the relative amount of target sequences in the probe mix, was analyzed by comparing the fluorescent intensity of corresponding individual spots after proper background subtraction. Positional information of clones on the arrays and the chromosomes was correlated. The ratios were plotted along individual chromosome for easy inspection. For each sample two experiments were performed: Cy5™-labeled nucleic acid (derived from tumor DNA) versus Cy3-labeled nucleic acid (derived from “normal” DNA) and Cy3-labeled nucleic acid (derived from tumor DNA) versus Cy5™-labeled nucleic acid (derived from normal DNA). Thus when the Cy5™ to Cy3™ ratios are plotted together along individual chromosome the two ratio curves looked reciprocal to each other. By performing two reciprocal experiments any ratio artifact can be easily identified.

Results:

In the above described studies, it was found that fluorescent signals were significantly stronger when the antioxidant dithiothreitol (DTT) was added to the hybridization buffer as compared to hybridization reactions lacking an antioxidant. Furthermore, the extent of “protection” against oxidation (i.e., stabilization of the fluorescent dye Cy5™) increased as the concentration of antioxidant increased (from 10 mM to 500 mM). For example, after 12 hours of hybridization, with use of DTT, the Cy5™ signal remained constant. After 48 hours of hybridization, the control (no antioxidant) sample showed a significant deterioration of the Cy5™ signal, i.e., enough of the Cy5™ had oxidized to a non-fluorescing state that no signal was detectable). In contrast, the DTT-containing sample remained relatively constant, and, in some samples, the level of Cy5™ fluorescence actually increased.

In the above described studies, it was also found that an “unbalanced” or “dynamically changing” humidity and/or temperature environments during hybridization significantly shortened the period of time needed to reach equilibrium between soluble labeled nucleic acid and immobilized probe nucleic acid.

If the humidity in the array hybridization chamber had the same concentration of hybridization buffer throughout the chamber, the humidity remained relatively constant throughout the chamber. More time was needed to reach equilibrium (between soluble nucleic acid and immobilized probe) under these constant conditions than under “dynamic” humidity conditions, i.e., an environment conducive to an imbalanced humidity environment, e.g., as the environment created as illustrated in FIG. 2. In this device, water was placed on one side of the array hybridization chamber and a 2× hybridization buffer was placed on the other side. Putting water on one side and 2× buffer on the other side generated a humidity gradient in the chamber. This resulted in an exchange of humidity around the four edges of the coverslip that unbalanced the humidity of the array hybridization chamber. The reaction chamber in FIG. 2 was also incompletely (i.e., only “loosely”) sealed to allow exchange of humidity with the outside environment.

While the invention is not limited by any particular mechanism of action, the dynamic humidity (and, similarly, dynamic temperature) conditions decrease the amount of self-association between the soluble, labeled nucleic acids; such self-association decreases their rate of hybridization to the immobilized probes on the array. Less self-association of soluble nucleic acid results in accelerated rate of association with immobilized probe, thereby decreasing the time needed to reach equilibrium.

Unbalanced humidity or temperature may also increase the movement of soluble sample to speed up the hybridization process. If the solution is relatively static, as is the case in an unchanging humidity (or temperature) environment, the mass transfer process is limited to a diffusion mechanism, which is extremely slow. Under slower, static conditions a significant amount of soluble nucleic acid fragments associates with other soluble nucleic acids before they have a chance to associate and hybridize to immobilized array target sites.

Hybridization efficiency (i.e., time to equilibrium) can also be enhanced by a hybridization environment that comprises changing hyper-/hypo-tonicity, e.g., a solute gradient. Thus, in alternative embodiments of the device, a solute gradient is created, and, in another embodiment, can be maintained throughout the hybridization reaction. In one exemplary device, a low salt hybridization solution can be placed on one side of the array hybridization chamber and a higher salt buffer (e.g., a 2× hybridization buffer) can be placed on the other side to generate a solute gradient in the chamber.

Hybridization efficiency (i.e., rate to equilibrium) was also greatly enhanced when the reaction chamber temperature was fluctuatingly modified by, e.g., an oven, or other device capable of creating changing temperatures, as compared to the rate observed using a controlled, constant temperature environment (the enhancing temperature change being more than the approximately +/− three degrees variation typical of most laboratory ovens).

A number of embodiments of the invention have been described. Nevertheless, it will be understood that various modifications may be made without departing from the spirit and scope of the invention. Accordingly, other embodiments are within the scope of the following claims.

What is claimed is:

1. A method for generating a molecular profile of genomic DNA by hybridization of a genomic DNA target to an immobilized nucleic acid probe, comprising the following steps:
(a) providing a plurality of nucleic acid probes comprising a plurality of immobilized nucleic acid segments;
(b) providing a sample of target nucleic acid comprising fragments of genomic nucleic acid labeled with a detectable moiety, wherein each labeled fragment consists of a length smaller than about 200 bases; and
(c) contacting the genomic nucleic acid of step (b) with the immobilized probes of step (a) under conditions allowing hybridization of the target nucleic acid to the probe nucleic acid.

2. The method of claim 1, wherein each labeled fragment consists of a length no more than about 150 bases.

3. The method of claim 2, wherein each labeled fragment consists of a length no more than about 100 bases.

4. The method of claim 3, wherein each labeled fragment consists of a length no more than about 50 bases.

5. The method of claim 4, wherein each labeled fragment consists of a length no more than about 30 bases.

6. The method of claim 3, wherein each labeled fragment consists of a length between about 30 bases and about 150 bases.

7. The method of claim 1, wherein the sample of target genomic nucleic acid is prepared using a procedure comprising random priming, nick translation, amplification, or equivalent, of a sample of genomic nucleic acid to generate segments of target genomic nucleic acid; followed by a step comprising fragmentation or enzymatic digestion, or both, of the segments to generate a sample of target genomic nucleic acid consisting of sizes smaller than about 200 bases.

8. The method of claim 7, wherein the random priming, nick translation, amplification, or equivalent, of the sample of genomic nucleic acid to generate segments of target genomic nucleic acid incorporates detectably labeled base pairs into the segments.

9. The method of claim 8, wherein the detectable label comprises Cy3™ or Cy5™ or equivalent.

10. The method of claim 1, wherein the sample of target genomic nucleic acid is prepared using a procedure comprising fragmentation of a genomic DNA to sizes smaller than about 200 bases by DNase enzyme, or equivalent, digestion of the segments.

11. The method of claim 1, wherein the sample of target genomic nucleic acid is prepared using a procedure comprising fragmentation of a genomic DNA to sizes smaller than about 200 bases by applying shearing forces sufficient to fragment genomic DNA followed by DNase enzyme, or equivalent, digestion of the sheared DNA.

12. The method of claim 1, wherein the conditions allowing hybridization of the target nucleic acid to the probe nucleic acid comprise stringent hybridization conditions.

13. The method of claim 12, wherein the stringent hybridization conditions comprise a temperature of about 60° C. to about 65° C.

14. The method of claim 1, wherein the target nucleic acid consists essentially of DNA derived from a human.

15. The method of claim 1, wherein the sample of target genomic nucleic acid comprises sequences representing a defined part of or substantially an entire chromosome.

16. The method of claim 15, wherein the sample of target genomic nucleic acid comprises sequences representing substantially an entire genome.

17. The method of claim 15 or 16, wherein the chromosomal or genome is derived from a human.

18. A composition comprising a sample of target nucleic acid comprising fragments of genomic nucleic acid labeled with at least one detectable moiety, wherein each labeled fragment has a length smaller than about 200 bases and the sample of labeled target genomic nucleic acid comprises sequences representing a defined part of or substantially a complete chromosome, or substantially a complete genome.

19. The composition of claim 18, wherein the target nucleic acid consists essentially of human DNA.

20. The composition of claim 18, wherein the chromosome or genome is mammalian DNA.

21. The method of claim 20, wherein the DNA is human DNA.

22. The composition of claim 18, wherein each labeled fragment consists of a length no more than about 100 bases.

23. The composition of claim 22, wherein each labeled fragment consists of a length no more than about 50 bases.

24. The composition of claim 23, wherein each labeled fragment consists of a length no more than about 50 bases.

25. The composition of claim 22, wherein each labeled fragment consists of a length between about 30 bases and about 100 bases.

26. The composition of claim 18, wherein the detectable label comprises Cy3™ or Cy5™ or equivalent.

27. A kit comprising a sample of target nucleic acid and printed matter, wherein the target nucleic acid comprises fragments of genomic nucleic acid labeled with a detectable moiety, wherein each labeled fragment consists of a length smaller than about 200 bases and the sample of labeled target genomic nucleic acid comprises sequences representing substantially an entire chromosome or genome; wherein the printed matter comprises instructions on hybridizing the sample of target nucleic acid to a nucleic acid array.

28. A method for hybridizing a sample of labeled nucleic acid targets to a plurality of nucleic acid probes, comprising the following steps:
(a) providing a sample of nucleic acid targets comprising fluorescent-labeled nucleic acid fragments and a plurality of nucleic acid probes, wherein the fluorescent label is sensitive to oxidation;
(b) contacting the nucleic acid target and nucleic acid probe of step (a) under conditions allowing hybridization of the sample with the probe, wherein the hybridization conditions comprise use of a hybridization solution comprising at least one antioxidant,
wherein the amount of antioxidant in the solution is sufficient to inhibit the oxidation of the fluorescent label under the hybridization conditions.

29. The method of claim 28, wherein the fluorescent label comprises Cy5™ or equivalent.

30. The method of claim 28, wherein the fluorescent dye comprises a rhodarnine, a fluorescein or an aryl-substituted 4,4-difluoro-4-bora-3a, 4a-diaza-s-indacene dye or equivalents.

31. The method of claim 28, wherein the antioxidant is present in the hybridization solution at a concentration of about 25 mM to about 1000 mM.

32. The method of claim 31, wherein the antioxidant is present in the hybridization solution at a concentration of about 50 mM to about 500 mM.

33. The method of claim 28, wherein the antioxidant comprises a mercapto-containing compound.

34. The method of claim 33, wherein the mercapto-containing compound comprises a 2-mercaptoethylamine, a thiol N-acetylcysteine, an ovothiol, a 4-mercaptoimidazole.

35. The method of claim 28, wherein the antioxidant comprises an antioxidant vitamin-containing compound.

36. The method of claim 35, wherein the antioxidant vitamin-containing compound comprises an ascorbic acid (Vitamin C) or a tocopherol (Vitamin E).

37. The method of claim 28, wherein the antioxidant comprises a propyl gallate.

38. The method of claim 28, wherein the antioxidant comprises a beta-carotene.

39. The method of claim 28, wherein the antioxidant comprises a butylated hydroxytoluene (BHT) or a butylated hydroxyanisole (BHA).

40. A composition comprising a sample of Cy5™-labeled nucleic acid or equivalent in a solution comprising at least one antioxidant.

41. The composition of claim 40, wherein the antioxidant is present in a hybridization solution at a concentration of about 25 mM to about 1000 mM.

42. The composition of claim 41, wherein the antioxidant is present in a hybridization solution at a concentration of about 50 mM to about 500 mM.

43. The composition of claim 40, wherein the antioxidant comprises a mercapto-containing compound.

44. The composition of claim 43, wherein the mercapto-containing compound comprises a 2-Mercaptoethylamine, a thiol N-acetylcysteine, an ovothiol, a 4-mercaptoimidazole.

45. The composition of claim 40, wherein the antioxidant comprises an antioxidant vitamin-containing compound.

46. The composition of claim 45, wherein the antioxidant vitamin-containing compound comprises ascorbic acid (Vitamin C) or a tocopherol (Vitamin E).

47. The method of claim 40, wherein the antioxidant comprises a propyl gallate.

48. The method of claim 40, wherein the antioxidant comprises a betacarotene.

49. The method of claim 40, wherein the antioxidant comprises a butylated hydroxytoluene (BHT) or a butylated hydroxyanisole (BHA).

50. A kit comprising a sample of fluorescent dye-labeled nucleic acid or equivalent in a solution comprising at least one antioxidant and printed matter, wherein the printed matter comprises instructions on using the fluorescent dye-labeled nucleic acid in a hybridization reaction with another nucleic-acid.

51. The kit of claim 50 further comprising a hybridization complex wash solution comprising at least one antioxidant.

52. The kit of claim 50, wherein the fluorescent dye comprises a Cy5™ or equivalent.

53. The kit of claim 50, wherein the fluorescent dye comprises a rhodamine, a fluorescein or an aryl-substituted 4,4-difluoro-4-bora-3a, 4a-diaza-s-indacene dye or equivalents.

54. A method for hybridizing a sample of nucleic acid targets to a plurality of immobilized nucleic acid probes, comprising the following steps:
(a) providing a sample of nucleic acid targets and a plurality of immobilized nucleic acid probes;
(b) contacting the nucleic acid target and nucleic acid probe of step (a) under conditions allowing hybridization of the sample with the probe, wherein the hybridization conditions comprise a controlled hybridization environment comprising an unsaturated humidity environment.

55. The method of claim 54, wherein the unsaturated humidity environment is controlled at about 90% humidity, about 80% humidity, about 70% humidity, about 60% humidity, about 50% humidity, about 40% humidity, about 30% humidity, or about 20% humidity.

56. The method of claim 54, wherein the humidity of the controlled environment is periodically changed during the hybridization of step (b).

57. The method of claim 56, wherein the humidity is periodically changed at about three hour intervals, at about two hour intervals, at about one hour intervals, at about 30 minute intervals, at about 15 minute intervals or at about 5 minute intervals, or a combination thereof.

58. The method of claim 54, wherein the hybridization conditions comprise a controlled temperature environment.

59. The method of claim 58, wherein the temperature of the controlled environment is periodically changed during the hybridization of step (b).

60. The method of claim 59, wherein the temperature is periodically changed at about three hour intervals, at about two hour intervals, at about one hour intervals, at about 30 minute intervals, at about 15 minute intervals or at about 5 minute intervals, or a combination thereof.

61. A composition comprising an array of immobilized nucleic acids in a housing, wherein the housing comprises a component to measure and control the humidity in the housing.

62. The composition of claim 61, wherein the housing further comprises a component to measure and control the temperature in the housing.

63. The composition of claim 62, wherein the housing further comprises a component that allows programmable or preset control of the humidity and the temperature.

64. An array of immobilized probe nucleic acids in a humidity-controlled housing, wherein the housing comprises a means to control the amount of humidity in the housing during hybridization of the probes to a target in an aqueous hybridization solution.

65. An array of immobilized probe nucleic acids in a humidity-controlled housing, wherein the housing comprises a humidifier component that can control the amount of humidity in the housing during contact of the probes to an aqueous hybridization solution.

66. A kit comprising an array of immobilized nucleic acids in a housing and printed matter, wherein the housing comprises a component to control the amount of humidity in the housing, a component to control the temperature in the housing, and a component to preset or program control of the humidity and the temperature, and the printed matter comprises instructions for presetting or programming conditions in the housing to hybridize a target to the immobilized nucleic acids of the array under controlled hybridization conditions that comprise fluctuation of humidity and temperature during a nucleic acid hybridization step.
